(19) United States
(12) Patent Application Publication
(10) Pub. No.: US 2011/0213277 A1
(43) Pub. Date: Sep. 1, 2011

(54) SYSTEM, METHOD, AND APPARATUS FOR OBJECTIVE ASSESSMENT OF MOTOR SIGNS AT THE EXTREMITIES

(75) Inventors: James McNames, Portland, OR (US); Lars Holstrom, Portland, OR (US); Joshua Booren, Portland, OR (US); Edward Katz, Portland, OR (US); Forest Kernan, Portland, OR (US); Brent Casady, Portland, OR (US); John Nutt, Portland, OR (US); Craig Kinnie, Portland, OR (US); Pedro Mateo Riobo Aboy, Portland, OR (US)

(73) Assignees: APDM, INC; PORTLAND STATE UNIVERSITY; OREGON HEALTH AND SCIENCE UNIVERSITY

(21) Appl. No.: 13/037,287

(22) Filed: Feb. 28, 2011

Related U.S. Application Data
(60) Provisional application No. 61/308,858, filed on Feb. 26, 2010.

(51) Int. Cl. A61B 5/11

(52) U.S. Cl. 600/595

(57) ABSTRACT

Disclosed embodiments include a system for objective assessment of motor signs at the extremities that comprises (a) a device for objective motor sign measurement, (b) a test protocol defining a prescribed repetitive activity, and (c) a signal processing and analysis system to generate one or more impairment metrics. According to a particular embodiment, the device for objective motor sign measurement is characterized by including means for producing a continuous measure of position of a limb or extremity during said prescribed repetitive activity during the entire movement.

### CROSS-REFERENCE TO RELATED APPLICATIONS

This application claims the benefit of U.S. Provisional Application No. 61/308,858 filed on 2010 Feb. 26, which is incorporated herein by reference.

### TECHNICAL FIELD

Disclosed embodiments relate to methods and apparatus for clinical assessment of movement disorders. Specifically, they relate to methods and apparatus for evaluating and characterizing the motor signs.

### BACKGROUND

Subjective assessment of movement disorders using clinical rating scales or poor instruments of mobility result in clinical trials that are inefficient, slow, complicated, and expensive. The primary outcomes are typically self-reported outcomes recorded from patient diaries (falls), clinician rating scales (UPDRS, Berg Balance scale), and/or patient questionnaires (PDQ-39). All of these instruments have limited resolution, are subjective, and are susceptible to bias. The UPDRS is coarse, subjective, momentary, stressful to the patient, and insensitive to subtle changes in the patient's motor state. It can only be applied in clinical settings by trained clinicians. To overcome the limitations of these instruments, clinical trials typically require a large number of subjects to detect a clinically significant difference between groups.

As an example of a movement disorder, Parkinson's disease (PD) is the second most common neurodegenerative disease and the most common serious movement disorder. It afflicts approximately 1 million people in the US alone, primarily people 60 years or older, costing the US economy over $25 billion annually. The most promising therapies for Parkinson's disease have the potential to slow the rate of disease progression. In clinical trials the effectiveness of these therapies is determined by regular assessments with a subjective rating scale every 3-9 months over periods ranging from 6 months to 2 years for each subject. Measures of functional motor impairment are necessary because there are no direct biological measures of the disease state. Since the natural rate of disease progression is slow and varies among people with Parkinson's, large trials with many subjects are required to determine if new therapies can slow or reverse disease progression. It is not known if precise objective measures of functional motor impairment could measure the rate of disease progression with greater sensitivity. This delays the production of new therapies that could slow or reverse Parkinson's disease progression. At the moment there is a lack of methods and instruments that are more accurate than rating scales for use in clinical trials of Parkinson's disease and other movement disorders. For instance, currently there are no validated instruments to measure functional motor impairment of Parkinson's disease in the upper and lower extremities. Novel and improved instruments that are more responsive to symptomatic interventions and can track disease progression more accurately than the motor section of the Unified Parkinson's Disease Rating Scale (UPDRS), which is the prevailing standard of functional motor impairment used in clinical trials today, are needed. Such instruments would reduce the number of subjects and cost of clinical trials, which in turn will accelerate the discovery, validation, and availability of new therapies.

### SUMMARY

Disclosed embodiments include a system for objective assessment of motor signs at the extremities that comprises (a) a device for objective motor sign measurement, (b) a test protocol defining a prescribed repetitive activity, and (c) a signal processing and analysis system to generate one or more impairment metrics. According to a particular embodiment, the device for objective motor sign measurement is characterized by including means for producing a continuous measure of position of a limb or extremity during said prescribed repetitive activity during the entire movement.

### BRIEF DESCRIPTION OF THE DRAWINGS

Disclosed embodiments are illustrated by way of example, and not by way of limitation, in the figures of the accompanying drawings:

FIG. 1 illustrates a block diagram of an embodiment of the system for objective motor sign measurement.

FIG. 2 illustrates an embodiment of a device for objective motor sign measurement.

FIG. 3 illustrates an embodiment of a device for objective motor sign measurement.

### DETAILED DESCRIPTION

In current medical practice and clinical trials involving various movement disorders assessment is conducted briefly using rating scales or less formal examinations. Disclosed embodiments include systems, methods, and apparatus for measuring the motor signs objectively and with greater precision than is possible with rating scales. Specifically, disclosed embodiments include systems comprising methods and apparatus for evaluating and characterizing the motor signs during prescribed repetitive activities with the hands, feet, and limbs. Disclosed embodiments are designed to measure the motor impairment caused by Parkinson's disease and other movement disorders. These embodiments can be used in clinical trials to evaluate the benefit of symptomatic therapies or therapies that are disease modifying and slow progression. The resulting overall impairment measure can be used to clinically determine which therapies are effective, measure the frequency and severity of motor fluctuations, and to optimize therapy on an individual basis. According to a particular embodiment, this can be accomplished systematically with n-of-1 trials.

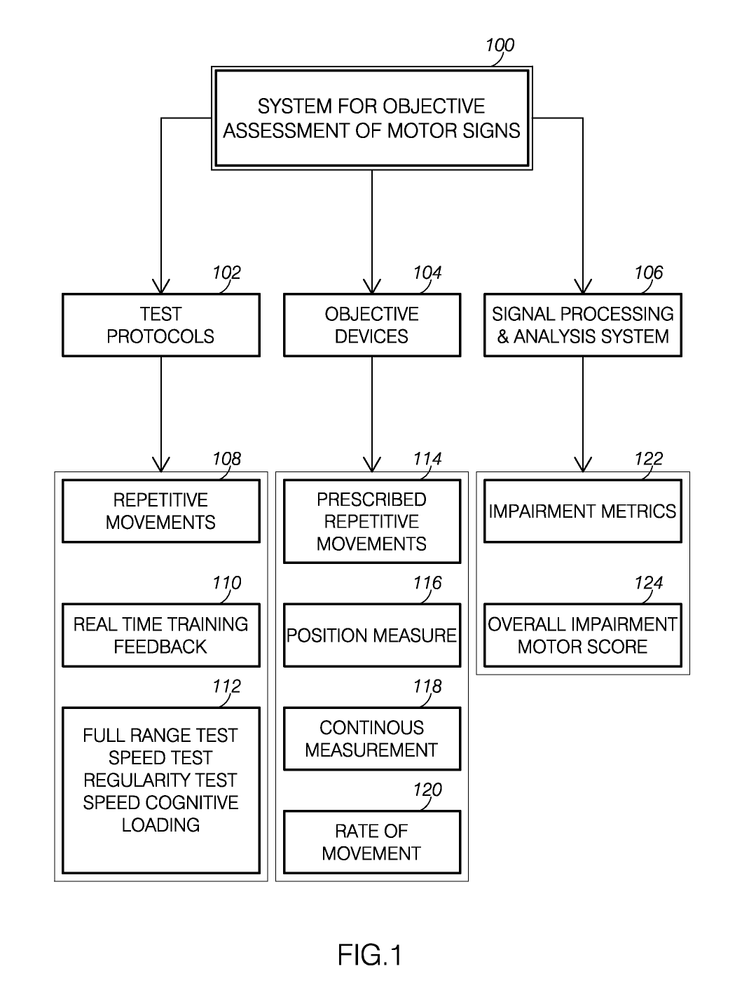

FIG. 1 illustrates a block diagram of a particular embodiment of the system 100 for objective motor sign measurement, without limitatation. Disclosed embodiments include a system for objective assessment of motor signs at the extremities, comprising: (a) a device for objective motor sign measurement 104, (b) a test protocol characterized by defining a prescribed repetitive activity 102, and (c) a signal processing and analysis system to generate one or more impairment metrics 106. According to a particular embodiment, the device for objective motor sign measurement is characterized by (a) being especially designed and adapted for prescribed repetitive movements 114, (b) producing a measure of position of a limb or extremity during the movement 116, and (c) measuring position continuously 118 during the entire movement. More particularly, the device for objective motor sign measurement can be a foot tapping device, a finger tapping device, a supination/pronation device, or a finger slider device. In one embodiment, the test protocol is further characterized by including real-time training and feedback 110 to the patient before and during a completion of the test protocol. Protocols 102 include tests for full range, speed, speed with cognitive loading 112, and regularity during repetitive movements 108. In a more particular particular embodiment the signal processing and analysis system 106 to generate one or more impairment metrics 122 further includes methods to generate a combined overall impairment motor score 124. Impairment metrics 122 include rate of movement, slowness of movement, hesitation, amplitude, regularity, and time variability. Overall impairment motor scores 124 are generated as summed subscores or using a linear or nonlinear model.

According to a particular embodiment, the test protocol 102 includes the instructions given to the subject prior to performing the task, any preparation or practice used to familiarize the subject with the device and test protocol. The device 104 includes one or more transducers to measure the position, force, or muscle activity during a prescribed activity. The signal analysis module 106 includes digital signal processing methods to process and analyze the signal recorded from the device in order to produce one or more measures of how well the subject performed the task. These metrics may be used directly in a clinical trial or by a clinician, or they may be used to calculate an overall measure of impairment.

### A. Devices for Objective Motor Sign Measurement

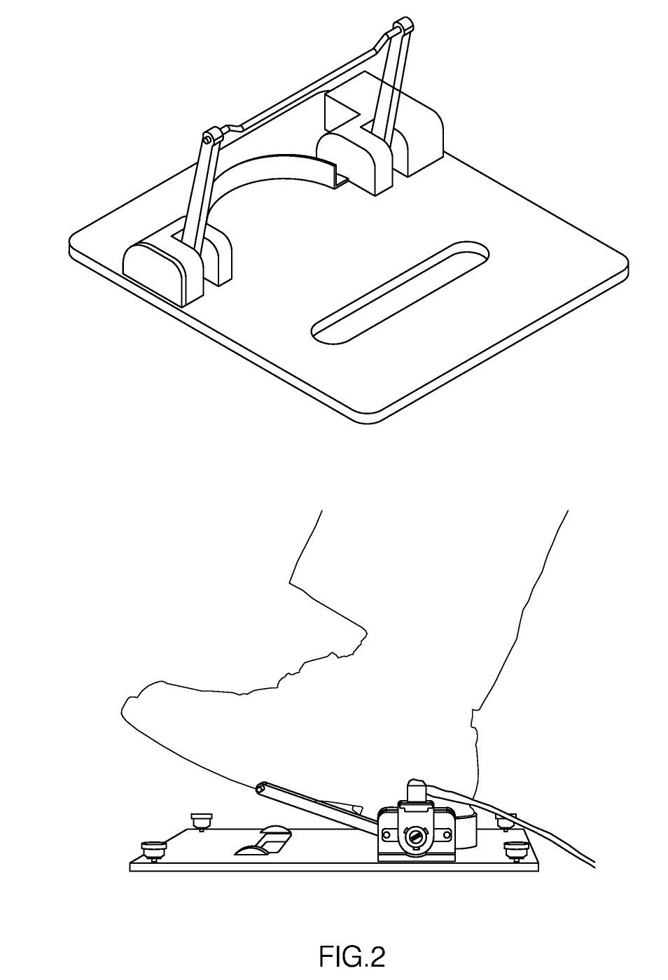

According to one embodiment, the devices 104 for objective motor sign measurement are characterized by: (a) being especially adapted for prescribed repetitive movements, (b) producing a measure of position of a limb or extremity during the movement, as opposed to a rate or acceleration, (c) measuring position continuously during the entire movement (previous devices are only able to measure the position at discrete points in time (this occurs, for example, with instruments that use buttons to measure how quickly a subject could tap their fingers), (d) measuring the rate at which a subject can move their limbs or other extremities, which is important because fine motor movement at the extremities is usually more impaired and easier to see visually than other movements that are more proximal, and (e) using one or more transducers to measure the position of an extremity or limb during the movement. FIG. 2 illustrates an embodiment of a device for objective motor sign measurement.

The following list describes specific embodiments of devices for objective motor sign measurement that share these common characteristics:

### Foot Tapping

According to one embodiment, the device is a foot tapping apparatus comprising a device for measuring the position, acceleration, or rotational rate of the foot during a foot tapping task. An example of such a device is shown in FIG. 2. In this case the subject taps their foot normally while the angle of the foot relative to the floor is recorded with a single transducer. According to one embodiment, the transducer is an optical encoder. In an alternative embodiment it is a potentiometer.

### Alternating Finger Tapping

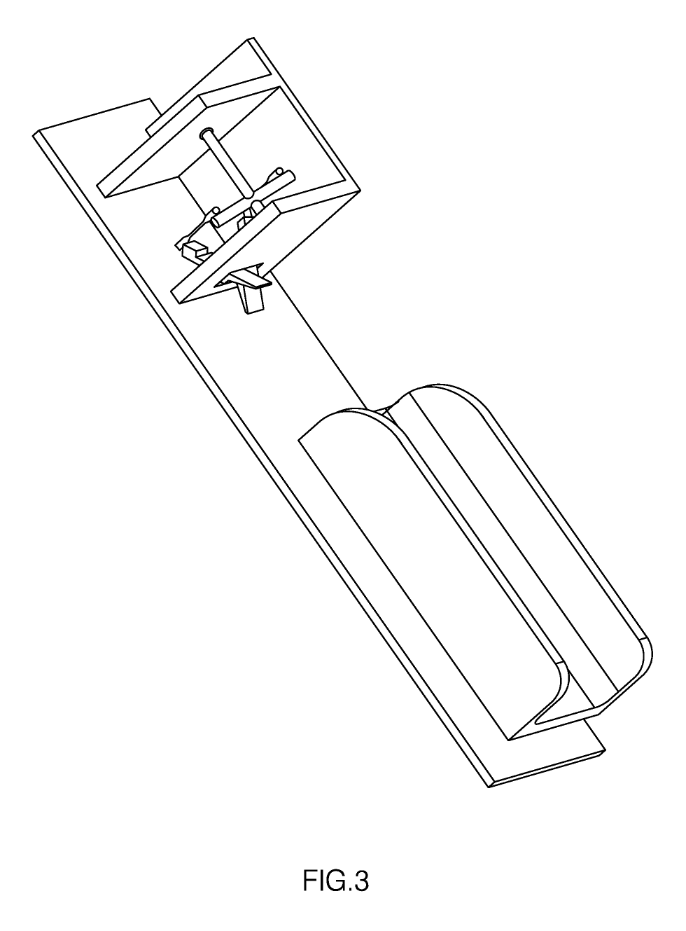

According to another embodiment the device for objective motor sign measurement is an alternating finger tapping apparatus. In a particular embodiment the device includes two keys similar to a piano keyboard that are used in a finger tapping test. The subject presses one key with their index finger and then pressed the other key with their middle finger, alternating back and forth between them. They keys are designed mechanically so that when one is pressed, the other rises. The position of the mechanically joined keys is then measured with a single encoder that may be optical or based on resistance, as with a potentiometer. FIG. 3 illustrates an embodiment of a device for objective motor sign measurement.

### Supination/Pronation

According to another embodiment the device for objective motor sign measurement is a supination/pronation device that measures the position of the forearm as the subject alternates between supination and pronation. According to a particular embodiment, the position of the forearm is recorded with a rotational transducer such as optical encoder or potentiometer. Depending on the particular embodiment, the interface between the rotational shaft and the hand may be in the form of a horizontal grip like the handlebars of a bicycle, may be spherical like a ball, may be a flat surface like a paddle, or may have a smooth shape comfortable for gripping like a door handle.

### Freehand Finger Tapping

According to another embodiment the device for objective motor sign measurement is a freehand finger tapper. According to a particular embodiment, the device consists of a rod that is attached to the index finger and slides through a fixture attached to the thumb that contains an encoder. Depending on the particular embodiment, the encoder may be optical or resistive. The device then produces a continuous measurement of the distance between the index finger and the thumb.

### Finger Slider

According to another embodiment the device for objective motor sign measurement is a finger slider. This device measures the position of a carriage that can slide back and forth horizontally between two points. The carriage is designed to moved by the hand using a finger or any type of grip.

### B. Protocols for Objective Motor Sign Measurement

All the protocols 102 are characterized by being designed and especially adapted for prescribed repetitive activities. According to particular embodiments the protocol allows for a period of training and for providing feedback to the patient. There are many variations on the test protocol that may be given to the subject. Particular embodiments of specific protocols, by way of example and not by way of limitation, are disclosed for illustrative purposes as follows.

### Full Range Protocol

According to one embodiment the subject is instructed to move back and forth as big as they can to measure the full range of motion. This is useful as an absolute measure because the range of motion is sometimes restricted in people with Parkinson's disease. It is also helpful to serve as a baseline that other measurements may be compared to.

Speed Protocol (as Fast as You can)

According to one embodiment the subject is instructed to perform the movement as fast as they can. To improve consistency, according to one embodiment, the subject is instructed to maintain a constant or minimal range of motion during this test. In one embodiment, the subject may be aided in this instruction by providing feedback on the range of motion through auditory, visual, or tactile cues that inform the subject when they have moved far enough and can begin moving back in the opposite direction.

Speed Cognitive Loading Protocol (as Fast as You can with Cognitive Loading).

People with Parkinson's disease often have a more difficult time performing two simultaneous tasks at the same time as compared to matched controls. Thus, according to one embodiment the subject is asked to perform the primary motor task at the same time as a secondary task that is essentially used to measure their ability to perform the task while distracted. The secondary task may be cognitive or physical. Common examples of cognitive tasks include counting backwards by 7 s starting at a number greater than 100, for example. Depending on the particular embodiment, it may also include naming tasks such as every word that begins with a certain letter or every object or place they can think of within a certain category, such as the names of all the states or every word that begins with the letter ‘b’. In another embodiment, it also includes of a common psychological test such as the strop test.

Regularity Protocol (as Regular as You can)

People with Parkinson's disease often have more frequent hesitations, halts, arrests, and episodes of freezing than matched controls. To ascertain this, according to one embodiment the subject subjects are instructed to perform the activity at a fixed pace as regular as they can.

### Worsening of Movement Over Time

The amplitude, rate, and variability of movement may worsen over the duration of a single test. According to one embodiment a metric of worsening impairment is calculated as the linear trend in amplitude over the duration of the test fitted with a least squares linear statistical model. According to another embodiment the average amplitude during the first part of the test is compared with the average amplitude during the last part of the test. In one embodiment, these are calculated for other metrics of impairment as well.

Worsening of Movement with Cuing Frequency

The movement timing, amplitude, and variability of movement may worsen as the frequency of the cuing is increased. According to one embodiment a metric of amplitude at a cuing frequency of 2.5 Hz is subtracted from the amplitude at a cuing frequency of 1.5 Hz. According to another embodiment a metric of amplitude at a cuing frequency of 2.5 Hz is divided by the amplitude at a cuing frequency of 1.5 Hz. According to another embodiment a linear model of the change in amplitude with frequency is fitted to the model to estimate the average change in impairment with frequency. These may be calculated for other metrics of impairment and other frequency combinations and ranges.

### Cued Movement Protocol

In one embodiment, the test is performed at a pace determined by an external cue similar to a metronome used by musicians. Depending on the particular embodiment, the cue may be auditory, visual, tactile, or any combination of the three. In one particular embodiment, the external cue is given at a range of different rates to assess how well the subject is able to respond.

### C. Signal Processing and Analysis Module

The apparatus includes a signal processing and analysis module 106 to generate one or more impairment metrics and a combined overall impairment motor score.

### C.1. Metrics for Objective Motor Sign Measurement

There are many ways in which Parkinson's disease and other movement disorders may affect the way someone can perform a repetitive task. These can be quantified in the form of metrics that are calculated from the signal obtained from the devices for objective motor sign measurement while the subject is performing the task. Particular embodiments of metrics, by way of example and not by way of limitation, are disclosed for illustrative purposes as follows.

### Rate of Movement

Parkinson's disease often affects how quickly subjects are able to move. The slowness of movement observed in people with Parkinson's disease is called bradykinesia. According to one embodiment, the metric is a rate of movement. The rate of movement of repetitive movements is calculated from the position signal either using time-domain techniques based on the detection of each discrete movement or using frequency domain techniques that measure the average frequency of movement. These are expressed either as a measure of the duration between tasks or the rate of movement. If the measure is determined from the discrete movements, the rate or duration is calculated with any measure of central tendency including the average, median, trimmed mean, or other common measures.

### Slowness of Movement

The rate of the movement may decrease during the course of the task. According to one embodiment, the metric measures the slowing of movement. According to a particular embodiment, this slowing of movement is calculated from the position signal using either time-domain techniques based on detection of each movement or using time-frequency domain techniques that continuously track the rate of the movement.

### Rate of Movement Variability

People with Parkinson's disease are known to perform repetitive tasks with greater variability in the rate of movement than matched controls. This especially occurs at higher frequencies. According to a particular embodiment, the variability of the rate of movement is calculated with either time-domain methods based on the detection of each movement or frequency-domain methods based on the distribution of signal power across frequency.

### Hesitation

People with Parkinson's disease often exhibit periods of halts, hesitations, arrests, or freezing during repetitive movements. According to one embodiment a hesitation and halts metric is calculated based on the period of time that the position is fixed either relative to the average rate of movement of the subject or in absolute terms. The hesitations and halts are quantified as either in terms of the duration of hesitations and halts, as the number of occurrences, or as the rate of occurrence.

### Amplitude

The amplitude of repetitive motions in Parkinson's disease is often decreased relative to matched controls. According to one embodiment, the amplitude of movement is calculated direction from the position signal based on the average range of movement during a complete cycle or an overage measure of the range of the signal. According to one embodiment, the amplitude is expressed in normalized units relative to the amplitude observed during another task or in absolute units, that may be either angular (e.g., degrees) or translational (e.g., meters).

People with Parkinson's disease may decrease the amplitude of movement during the course of a repetitive motion. The amplitude will also often decrease as the rate of movement is increased, either voluntarily or through cues. According to one embodiment, the decrease in amplitude is calculated either based on time-domain detection of each movement and calculation of how the amplitude of movement decreases or through direct estimation of how the envelope of the repetitive motion is diminished over the course of the task, using, for example, Hilbert transform techniques.

### Regularity

The amplitude of repetitive movements is often less regular in people with Parkinson's disease. According to an embodiment, the variability of the amplitude during a repetitive task may be calculated using any standard statistical measure of variation including the standard deviation and interquartile range. According to an alternative embodiment, a measure of signal regularity is employed such as Lempel-Ziv complexity, Approximate Entropy, Sample Entropy, or Multiscale Entropy.

### Time Variability

During cued tasks, one can also measure how consistently each movement occurs relative to the time of the external cue. According to a particular embodiment, this is calculated in normalized units relative to the cue period or in units of degrees as a measure of what is sometimes called phase variability.

### Cued Task

During cued tasks, one can measure the difference between the rate of movement and the cued rate. People with Parkinson's disease are known to perform repeated movement tasks faster than the cued rate, and especially at frequencies of 2 Hz or above. According to a particular embodiment the rate of error is calculated.

### C.2. Objective Motor Score

The apparatus includes signal processing and analysis means whereby one or more of the metrics are combined to calculate an overall score for the task that quantifies the overall impairment of the subjects' limb or extremity. According to a particular embodiment, the metrics are combined from different tasks and different devices to produce an overall measure of impairment. These combined measures of impairment are especially useful in clinical trials where it is important to have a scalar measurement that can be used to determine whether a therapy is effective or not. Particular embodiments disclosed herein by way of example include summed subscores, linear models, and nonlinear models.

### Summed Subscores

According to a particular embodiment the overall objective motor score is calculated based on each metric or task. In a particular embodiment, it is calculated as a z score from a population of control subjects or a typical population of people with Parkinson's disease. In other embodiments, the subscore is calculated based on any other form of normalization. The subscores are then added to calculate an overall score of impairment.

### Linear Models

According to a particular embodiment the overall objective motor score is calculated based on combining the metrics with a linear model that calculates a weighted combination of selected metrics. According to one embodiment, the metrics are selected and weighted empirically to optimize some measure of performance. Depending on the embodiment, the measure of performance may be based on one or more clinimetric criteria such as test-retest reliability, ability to distinguish between matched controls and people with Parkinson's disease, correlation with other instruments to measure motor signs, ability to distinguish between subjects with and without therapy, or the responsiveness to known therapies. The linear model may either combine the metrics directly or combine the metrics after a nonlinear operation, such as a squashing function that prevents exceptional performance from having too much influence on the overall score.

### Nonlinear Models

According to a particular embodiment the overall objective motor score is calculated based on combining the metrics with a nonlinear model. The model is optimized for metric selection and weighting using the same aforementioned criteria.

### D. Advantages of the Disclosed Objective Motor Sign Measurement System

According to one embodiment, the above disclosed method and apparatus are incorporated into an integrated Tapping Assessment Proficiency (TAP) system. The Tapping Assessment of Proficiency (TAP) that measures functional motor impairment of Parkinson's disease in the upper and lower extremities. The TAP is designed to be more responsive to symptomatic interventions and can track disease progression more accurately than the motor section of the Unified Parkinson's Disease Rating Scale (UPDRS), which is the prevailing standard of functional motor impairment used in clinical trials today.

### Track Functional Progression

The UPDRS has been the most rigorously and thoroughly evaluated for its clinimetric properties and especially its reliability and validity. However, the UPDRS is coarse, subjective, momentary, stressful to the patient, and insensitive to subtle changes in the patient's motor state. It can only be applied in clinical settings by trained clinicians. The TAP is designed to be more accurate, can track functional progression, can be self-administered, and is cost effective.

### Greater Reliability and Sensitivity

The slow rate of disease progression and poor sensitivity of the UPDRS necessitate large, cost-prohibitive clinical trials of therapies that may slow disease progression. Trials of therapies that may slow the rate of disease progression are difficult to design, require many subjects, must be conducted over a long period of time, and are expensive. For example, the recent ADAGIO trial included 1176 subjects from 129 centers in 14 countries to evaluate the potential of rasagiline, a monoamine oxidase type B (MOA-B) inhibitor, to slow the rate of disease progression. The TAP is designed to be an easy-to-use objective measure of the functional motor impairment with greater reliability and sensitivity would have permitted more frequent measurements and fewer subjects.

### Reduce the Placebo Effect

The placebo effect frequently observed in clinical trials can mask the actual benefit of the therapy. This has confounded many clinical trials, as occurred in a recent clinical trial of CERE-120. This is an adeno-associated virus (AAV) vector that carries the gene for the protein neurturin (NTN), a neurotrophic factor which enhances function in dopamine-secreting neurons. The results of a Phase 2 clinical trial of this therapy were recently announced. The trial included 58 subjects. The primary endpoint was improvement in the UPDRS motor score in the off state at a 12 month follow up as compared to a control group who received a sham surgery. Both groups showed approximately 7 points of improvement as compared to the baseline. There are many possible causes of such a dramatic placebo effect including enthusiasm of the participants and raters for the potential of this new therapy. The TAP is designed to be less susceptible to this enthusiasm, and therefore are likely to measure the objective changes in motor function with various interventions.

### Greater Reliability

Biomechanical instruments have excellent reliability because they lack the variability caused by the subjective judgment and scoring of prescribed movements used in rating scales. This does not eliminate the variability in how the subject performs the task, but it eliminates the variability in the rating of the task. The reliability is also improved in systems that deliver clear, consistent instructions and are mechanically designed to ensure the prescribed movement is performed consistently every time. Ultimately, if the task is simple enough to use in the home, the reliability could be improved further by averaging the fluctuations that occur from day-to-day over several weeks to obtain a more accurate overall measure of impairment.

### Greater Precision

Most rating scales have a resolution of 3-7 points. The motor UPDRS uses a 5 point scale to rate each task. Systems for objective measures use floating point numbers with very fine precision to represent the score of each task. Individual performance metrics can also be combined to result in an overall motor performance score that is sensitive to functional progression. The TAP system is designed to have significantly greater resolution than a 3-7 point scale.

### Reduced Variation

It is impractical to ask subjects to perform each task in a rating scale more than once because the scale is too coarse and raters are too unreliable for repeated tasks to add any value. With objective measures, the performance can be averaged over several trials to determine the average performance that is insensitive to variation between trials. This improves both the reliability and precision of objective measures, as compared to rating scales. Repeated trials can also help eliminate the effects of outliers.

### Less Number of Subjects

More accurate instruments of functional impairment could reduce the number of subjects and cost of clinical trials and accelerate the discovery and validation of new therapies. An instrument of motor function with greater reliability and responsiveness could measure the rate of functional progression more accurately than the UPDRS. This could significantly reduce the number of subjects, complexity, and cost of clinical trials of therapies that may slow the rate of disease progression.

While particular embodiments have been described, it is understood that, after learning the teachings contained in this disclosure, modifications and generalizations will be apparent to those skilled in the art without departing from the spirit of the disclosed embodiments. It is noted that the foregoing embodiments and examples have been provided merely for the purpose of explanation and are in no way to be construed as limiting. While the system has been described with reference to various embodiments, it is understood that the words that have been used herein are words of description and illustration, rather than words of limitation. Further, although the system has been described herein with reference to particular means, materials and embodiments, the actual embodiments are not intended to be limited to the particulars disclosed herein; rather, the system extends to all functionally equivalent structures, methods and uses, such as are within the scope of the appended claims. Those skilled in the art, having the benefit of the teachings of this specification, may effect numerous modifications thereto and changes may be made without departing from the scope and spirit of the disclosed embodiments in its aspects.

The invention claimed is:

1. A system for objective assessment of motor signs at the extremities, comprising:
(a) a device for objective motor sign measurement;
(b) a test protocol defining a prescribed repetitive activity; and
(c) a signal processing and analysis system to generate one or more impairment metrics.

2. The system of claim 1, wherein said device for objective motor sign measurement includes means for producing a continuous measure of position of a limb or extremity during said prescribed repetitive activity during the entire movement.

3. The system of claim 2, wherein said device for objective motor sign measurement is a foot tapping device.

4. The system of claim 2, wherein said device for objective motor sign measurement is a finger tapping device.

5. The system of claim 2, wherein said test protocol further includes real-time training and feedback to a patient before and during a completion of said test protocol.

6. The system of claim 5, wherein said test protocol is a full range protocol, a speed protocol, a speed cognitive loading protocol, or a regularity protocol.

7. The system of claim 2, wherein said signal processing and analysis system to generate one or more impairment metrics further includes methods to generate a combined overall impairment motor score.

8. The system of claim 2, wherein said one or more impairment metrics include a rate of movement metric.

9. The system of claim 2, wherein said one or more impairment metrics include a slowness of movement metric.

10. The system of claim 2, wherein said one or more impairment metrics include a rate of movement variability metric.

11. The system of claim 2, wherein said one or more impairment metrics include a hesitation.

12. The system of claim 2, wherein said one or more impairment metrics include an amplitude metric.

13. The system of claim 2, wherein said one or more impairment metrics include is a regularity metric.

14. The system of claim 2, wherein said one or more impairment metrics include a time variability metric.

15. The system of claim 2, wherein said one or more impairment metrics include a worsening of movement over time metric.

16. The system of claim 2, wherein said one or more impairment metrics include a worsening of movement with cuing frequency.

17. The system of claim 2, wherein said regularity metric is based on a signal regularity measure chosen from the group consisting of Lempel-Ziv complexity, Approximate Entropy, Sample Entropy, and Multi-Scale Entropy.

18. The system of claim 7, wherein said overall impairment motor score is based on a summed of subscores.

19. The system of claim 7, wherein said overall impairment motor score is based on a linear model.

20. The system of claim 7, wherein said overall impairment motor score is based on a nonlinear model.
